(19) United States
(12) Patent Application Publication
(10) Pub. No.: US 2010/0129427 A1
(43) Pub. Date: May 27, 2010

(54) Hemostatic Wound Dressings

(75) Inventors: John Hen, Bradenton, FL (US); Talmadge Kelly Keene, Apollo Beach, FL (US); Mark Travi, Venice, FL (US)

Correspondence Address: CHARLES J. PRESCOTT, P.A., SUITE 115, 2033 WOOD STREET, SARASOTA, FL, 34237, US

(73) Assignee: BIOLIFE, L.L.C., Sarasota, FL (US)

(21) Appl. No.: 12/626,478

(22) Filed: Nov. 25, 2009

Related U.S. Application Data
(60) Provisional application No. 61/200,128, filed on Nov. 25, 2008.

(51) Int. Cl. A61L 15/16

(52) U.S. Cl. 424/445

(57) ABSTRACT

Hemostatic wound dressings for substantially arresting the flow of severe, life threatening bleeding from a wound by rapidly adhering to the wound site, absorbing and concentrating and thickening the blood at the dressing blood interface and accelerating the natural clot formation beneath the dressing and finally, forming a strong seal that will substantially prohibits further flow of blood out of the wound site. These hemostatic wound dressings are formed of unique combinations of hemostatic dressing aspects which achieve wound seal strengths that are significantly higher than the sum of seal strengths expected from the individual aspects alone. Some embodiments also achieve these synergistic seal strengths by combining one hemostatic dressing with a non-hemostatic device.

### CROSS-REFERENCE TO RELATED APPLICATIONS

Not applicable

### STATEMENT REGARDING FEDERALLY SPONSORED RESEARCH OR DEVELOPMENT

Not applicable

### INCORPORATION-BY-REFERENCE OF MATERIAL SUBMITTED ON A COMPACT DISC

Not applicable

### BACKGROUND OF THE INVENTION

1. Field of the Invention

This invention relates generally to wound care and hemostatic dressings and more particularly to a novel topically applied dressing composition that dramatically arrests traumatic and severe bleeding in wounds and provides wound-healing properties.

2. Description of Related Art

Hemostatic agents are well known in the prior art. Patterson et al., U.S. Pat. No. 6,187,347, discloses a free flowing powder to arrest bleeding from a wound comprising the steps of providing a substantially anhydrous compound of a salt ferrate which will hydrate in the presence of blood to clot blood and produce oxygen and applying said compound to the wound for a time sufficient to arrest blood flow and substantially reduce the microbial population by the presence of oxygen and forming a protective coating over the wound. In one embodiment, a cation exchange material is mixed with the salt ferrate to provide a protective coating over the wound for protection and enhanced healing. The salt ferrate provides the oxygen required to substantially reduce the level of bacteria, virus and fungus at the wound site.

In military and civilian trauma units, the use of cotton gauze pads capable of absorbing 250 ml of blood are known as passive dressings for controlling bleeding at active bleeding wound sites such as an external hemorrhage. Cotton gauze pads are considered passive since they do not initiate or accelerate blood clotting. Cellulose hemostatic compositions have been disclosed in U.S. Pat. Nos. 2,914,444 and 3,122,479. U.S. Pat. No. 4,626,253 teaches the preparation of surgical hemostats made from a knitted fabric of oxidized cellulose having superior handling and hemostatic properties.

Faster hemostasis is provided by the use of calcium-modified oxidized cellulose as taught by Stilwell et al. U.S. Pat. No. 5,484,913. Cochrum et al. in U.S. Pat. No. 7,101,862 describe an article that promotes hemostasis that contains cellulose and a polysaccharide covalently linked to the cellulose. U.S. Pat. No. 6,652,840 by Prevendar discusses a bleeding control composition consisting of regenerated oxidized cellulose, ferric sulfate, aluminum chloride, aluminum ammonium sulfate, absorbable gelatin and a solvent. U.S. Pat. No. 7,279,177 by Looney et al. describes a hemostatic wound dressing that utilizes a fibrous, fabric substrate made from carboxylic-oxidized cellulose with a biocompatible, water soluble or water swellable cellulose polymer distributed within the fabric. Zhang et al. in U.S. Pat. No. 7,262,181 teach that hemostatic materials can be made of water-soluble cellulose ether derivatives, such as methylcellulose, ethylcellulose and hydroxyethylcellulose. Improved hemostatic wound dressings can be made from distributing hydroxyethyl cellulose uniformly into a piece of absorbable hemostat based on oxidized regenerated cellulose as discussed in U.S. Pat. No. 7,019,191 by Looney et al.

Alginates and chitosan have long been known as effective hemostatic wound dressings. A synergistic combination of alginate and chitosan with high absorption capacity provide better hemostatic properties as discussed by Pandit in U.S. Pat. No. 5,836,970.

Current hemostatic agents absorb blood to different degrees but have limited ability to stop bleeding. A hemostatic dressing has yet to be developed that controls traumatic and uncontrolled bleeding while cutting down the time to hemostasis substantially.

The foregoing examples of the related art and limitations related therewith are intended to be illustrative and not exclusive. Other limitations of the related art will become apparent to those skilled in the art upon a reading of the specification and a study of the drawings.

### BRIEF SUMMARY OF THE DISCLOSURE

This disclosure is directed to improved hemorrhage control wound dressings and the methods of applying such dressings. The wound dressing is capable of substantially stopping the flow of severe, life-threatening bleeding from the wound by rapidly adhering to the wound site, absorbing, concentrating and thickening the blood at the dressing/blood interface, accelerating the natural clot formation beneath the dressing and forming a strong seal that substantially prohibit the flow of blood out of the wound site. The novel dressing includes a combination of hemostatic dressings which achieve seal strengths (defined by mm pressure to failure) that are significantly higher than the sum of seal strengths expected from the individual components. In some cases, the increase in seal strengths is achieved by combining one hemostatic dressing with a non-hemostatic device such as a polyurethane foam.

It is therefore an object of this invention to provide a wound dressing which is capable of substantially stopping the life threatening flow of blood from a wound.

Still another object of this invention is to provide a wound dressing which accelerates the natural blood clot formation from a severely bleeding wound.

Yet another object of this invention is to provide a wound dressing capable of forming a strong seal over the wound to substantially inhibit blood flowing from the wound site.

The following embodiments and aspects thereof are described and illustrated in conjunction with systems, tools and methods which are meant to be exemplary and illustrative and not limiting in scope. In various embodiments one or more of the above-described problems have been reduced or eliminated while other embodiments are directed to other improvements. In addition to the exemplary aspects and embodiments described above, further aspects and embodiments will become apparent by reference to the drawings and by study of the following descriptions.

### BRIEF DESCRIPTION OF THE SEVERAL VIEWS OF THE DRAWING(S)

FIG. 1 is a perspective view of one embodiment of the invention.

FIG. 2 is a section view in the direction of arrows 2-2 in FIG. 1.

FIG. 3 is a perspective view of a second embodiment of the invention.

FIG. 4 is a section view in the direction of arrows 4-4 in FIG. 3.

FIG. 5 is a perspective view of a third embodiment of the invention.

FIG. 6 is a section view in the direction of arrows 6-6 in FIG. 5.

FIG. 7 is a perspective view of a fourth embodiment of the invention.

FIG. 8 is a section view in the direction of arrows 8-8 in FIG. 7.

FIG. 9 is a perspective view of a fifth embodiment of the invention.

FIG. 10 is a section view in the direction of arrows 10-10 in FIG. 9.

FIG. 11 is a perspective view of a sixth embodiment of the invention.

FIG. 12 is a section view in the direction of arrows 12-12 in FIG. 11.

FIG. 13 is a top plan view of a seventh embodiment of the invention.

FIG. 13A is a section view in the direction of arrows 13A-13A in FIG. 13.

FIG. 14 is a simplified section view of a test fixture used in in vitro hemostasis test apparatus.

FIG. 15 is a simplified schematic view of the in vitro hemostasis testing system incorporating the test fixture of FIG. 14.

FIG. 16 shows the hemostasis test fixture of FIG. 14 incorporating a weight applied over the hemostasis agent being tested.

Exemplary embodiments are illustrated in reference figures of the drawings. It is intended that the embodiments and figures disclosed herein are to be considered to be illustrative rather than limiting.

### DETAILED DESCRIPTION OF THE INVENTION

The following provides a general description of each of the aspects or elements of the present invention utilized to formulate the various embodiments of the invention described below and with respect to the drawings.

### Material A

The water soluble or water-swellable, adhesive hemostatic dressing, material or pouch A can be a knitted, non-woven or woven fabric made from oxidized cellulose, poly-vinylpyrrolidone or its copolymers, alginate, crosslinked polyvinyl alcohol or copolymers of polyvinylalcohol. Desired physical properties of A include: high water absorption, high swellability, strong wet-strength and instant strong adherence to the wound site.

The oxidized celluloses include but are not limited to: regenerated etherized and oxidized natural fiber cellulose, carboxylic-oxidized cellulose, carboxy methyl cellulose, hydroxyl ethyl cellulose, methyl cellulose, ethyl cellulose, hydroxyl propyl cellulose, methyl hydroxyl propyl cellulose and methyl, hydroxyl ethyl cellulose. An embodiment includes blends of oxidized cellulose such as mixtures of hydroxyl propyl cellulose, methyl hydroxyl propyl cellulose, and methyl hydroxyl ethyl cellulose. The oxidized celluloses can be post treated with a water sensitive coating of polyvinylpyrrolidone or polyacrylic acid or sodium alginate to improve its adhesive properties to increase adhesion to skin or human tissue.

### Material B

Hemostatic Powder (B) includes any powder material that exerts a passive or active mode of action in hemostasis. An example of a passive hemostatic powder is a blend of potassium ferrate and the acid form of a cross-linked ion exchange resin (copolymer of polystyrene and divinylbenzene) known as PRO QR Powder as described in U.S. Pat. No. 6,187,347. Other passive hemostatic powder can include microporous, controlled porosity polysaccharide (such as HEMADERM or TRAUMADEX), ethyl cellulose beads and beads of other oxidized celluloses, powder with blend of maltodextrin and other hemostatic agents (an example is deRoyal Multidex Hydrophilic Powder Wound Dressing which is a blend of maltodextrin, sodium alginate and chitosan), powder composed of calcium and sodium alginates with or without chitosan, granular zeolite with strong absorption and exothermic hydration properties such as QUIKCLOT, clays such as bentonite (also known as smectites or montmorillonite).

### Material C

Water absorbing material (C) in powder form may include any highly hydrophilic materials whether natural or synthetic. Examples include super absorbing polymers (SAP) (salt of crosslinked polyacrylic acid and its copolymers; vitrified starch such as Safe and Natural Absorbent Polymers), synthetic ion exchange resins (weak acid and strong acid cation organic exchange resin, anion organic exchange resin), inorganic ion exchange resins including zeolites, and absorbents such as clays.

### Material D

Water insoluble pouch (D) includes polymer mesh prepared from hydrophobic polymers such as polypropylene and other polyolefins, polyamides, polyesters, polyurethanes and mixtures thereof.

### Material E

Moisture absorbing backing (E) include any foam or solid sheet material that can absorb moisture or blood easily and provide back pressure to reinforce A. For example, if A was a regenerated and oxidized cellulose non-woven fabric, the oxidized cellulose would wet quickly in the presence of blood and strongly adhere to the wound site and surrounding tissues. The backing foam (E) wicks the blood away and provides strength to oxidized cellulose which normally would quickly turn into a gel without E resulting in rebleeding or continued blood loss.

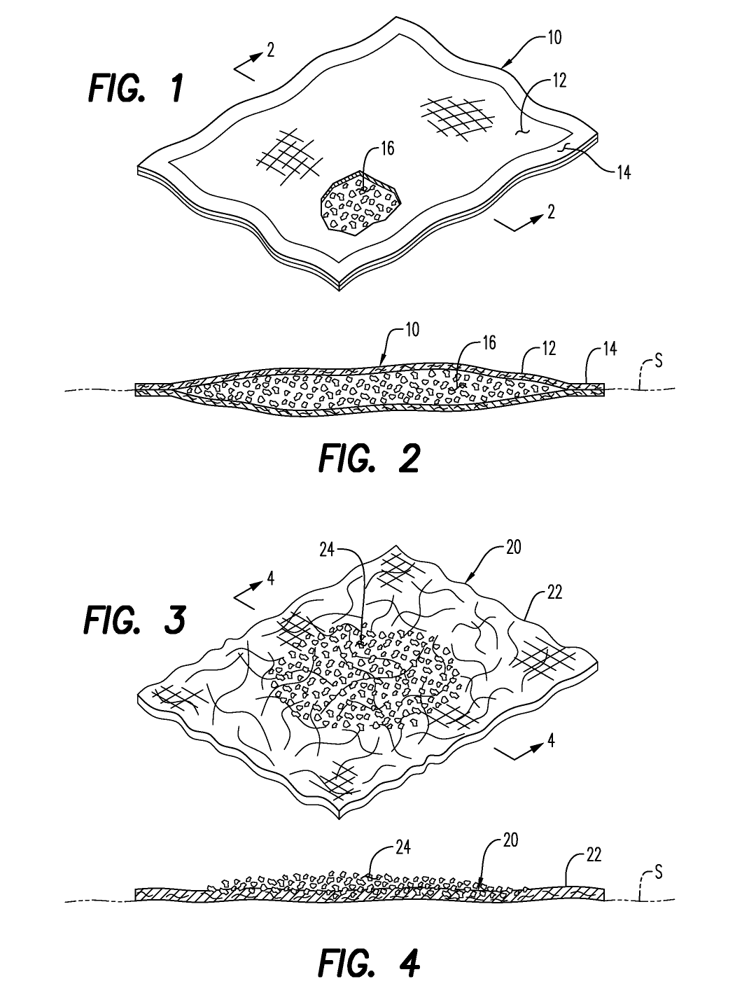

The following types of wound dressings are preferred combinations of the above aspects of this invention:
1. A water soluble or water-swellable, adhesive hemostatic pouch (A) containing a hemostatic powder (B) (FIGS. 1 and 2); or the powder B on top of a gauze of material A (FIGS. 3 and 4).

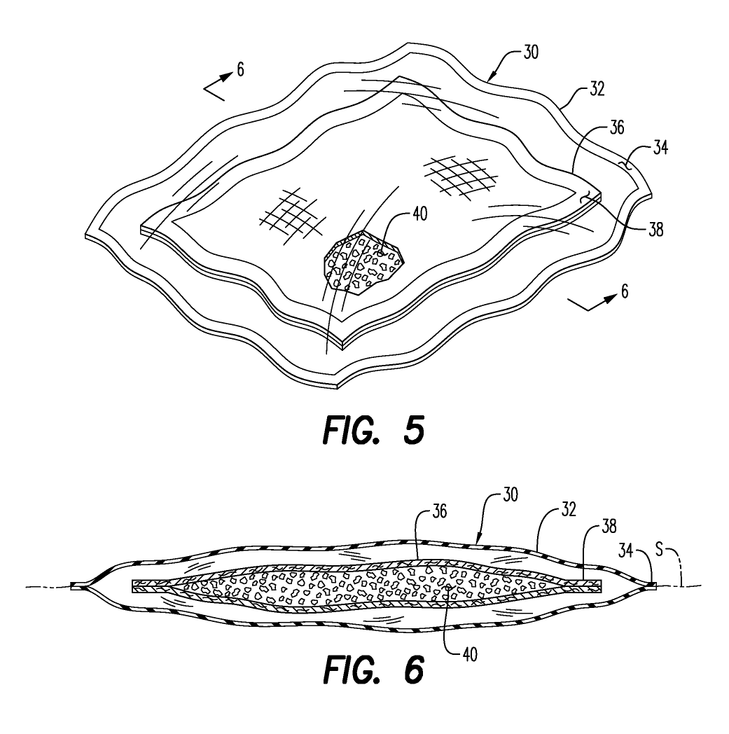

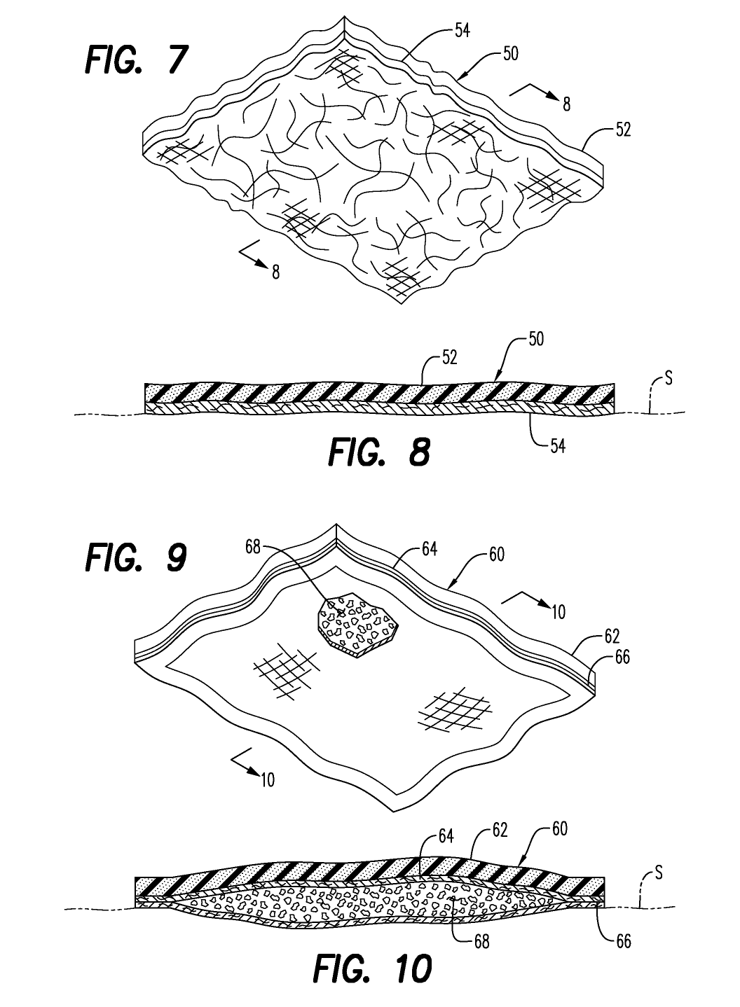

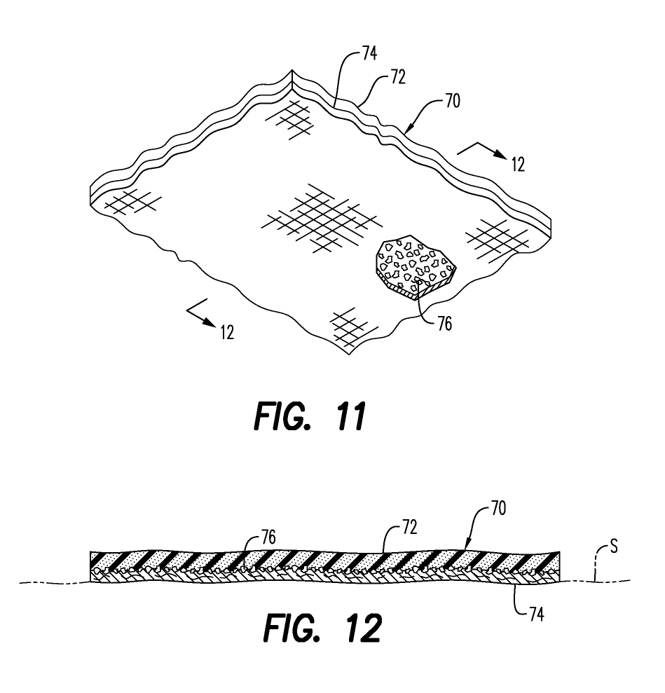

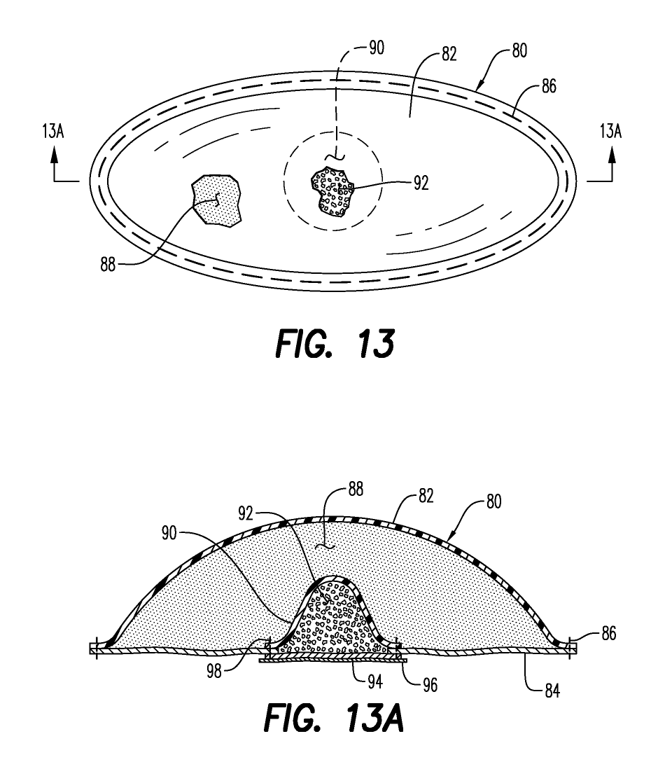

2. Pouch A containing water absorbing material (C) (FIGS. 1 and 2); the dressing can simply be material C on top of a gauze of same material as pouch A.
3. Hemostatic powder B in water-insoluble pouch (D) wherein D is placed inside the pouch A (FIGS. 5 and 6).
4. Material A in the form of a gauze backed by a moisture absorbing backing (E) such as a hydrophilic polyurethane foam (FIGS. 7 and 8); or, Pouch A containing material E (FIGS. 9 and 10).
5. A three-layer dressing including (FIGS. 11 and 12):
   a. top layer of water soluble/swellable adhesive material A
   b. intermediate layer of hemostatic powder B or water absorbing material C
   c. bottom layer of moisture absorbing backing E
6. An impermeable outer pouch (Material D) containing super absorbing polymer (SAP) with a hemostatic powder B separated from the SAP by an inner moisture impermeable pouch (also formed of Material D) having a lower surface formed of Material A to deploy Material B to the wound site as the bulk of the blood is absorbed through a wicking lower surface of pouch by the SAP (FIGS. 13 and 13A).

### Methods of Preparing Etherized Celluloses

A number of examples of test results using this test apparatus and procedure are as follows:

### Method 1

One method of making etherized celluloses includes pre-washing the cellulose gauze or fabric in an ethanol or similar solution. Next the gauze is placed in an aqueous solution of sodium or potassium hydroxide at 20 C to 50 C for 1 to 4 hours to break down the celluloses and add OH bases to the molecules. The gauze is then treated with one or more halogenated alkyl compounds, such as methyl chloride, ethyl chloride, and propyl chloride, chloroacetic acid, chloropropanoic acid and chlorobutanoic acid, among others. Additional alkenyl oxides may also be used, such as ethylene oxide and propylene oxide, among others. The mixture is heated at a temperature from 50 C to 160 C. for about 2-6 hours. The product is then neutralized with C₁ to C₅ lower alkyl alcohols which include methanol, ethanol, propanol, butanol, pentanol, and isopropyl alcohol, together with acids such as acetic acid or phosphoric acid, to a pH of about 5-8.

### Method 2

Another method of preparing etherized celluloses is to treat a medical grade absorbent gauze first in an alcoholic solution followed by treating the gauze in an aqueous or ethanolic, strongly alkaline solution at 20 to 50 C for a specified time. Next the gauze is treated in a solution of acetic chloride (acetic acid, acetic acid salt or any acid or acid salt with a carboxylic acid group in the molecule can be used in place of acetic chloride) at a concentration of 20-80% in an ethanol solution for 2-6 ours at 20 C to 80 C. This step produces carboxy methyl cellulose converting the gauze to a soluble hemostatic wound dressing. The resulting alkaline gauze is washed with several ethanol washes to remove the alkalinity and the gauze is dried in the oven, packaged and sterilized.

### Method 3

Yet another method of preparing a water-soluble, hemostatic gauze matrix includes the steps of mixing one or more of the etherized cellulose compounds, (typically produced as described hereinabove), and a hemostatic compound in a non-aqueous solvent such as ethanol to form a fibrous pulp, said hemostatic compound typically comprising chitosan, one or more water-soluble polysaccharide gums, and one or more surfactants.

After ensuring a substantially even dispersion of the mixture, the fibrous pulp is collected on forming fabric such as used in paper manufacturing to allow drainage of the pulp solution while retaining the fibers. The fibrous pulp is collected onto the forming fabric under vacuum conditions. The collected wet pulp undergoes compression and freeze drying to produce a sponge.

Referring now to the drawings, and firstly to FIGS. 1 and 2, a first embodiment of the invention is there shown generally at numeral 10 and includes a water soluble or water-swellable hemostatic pouch 12 containing a hemostatic powder 16 sealed therewithin along sealed margins 14 of the pouch 12. The pouch 12, as previously discussed, is preferably formed of Material A, while the hemostatic powder is preferably formed of Material B. The pouch 12 is preferably formed to be symmetric so that it may be applied on either side thereof against the surface of skin S. This embodiment 10 may also be formed having the water absorbing Material C in place of Material B.

Referring now to FIGS. 3 and 4, a second embodiment of the invention is there shown generally at numeral 20 and includes a quantity of gauze 22 formed of Material A atop which a quantity 24 of powder B is applied and enmeshed into the fibers of the gauze 22. The unpowdered surface of the gauze 22 is applied against the skin S covering the wound.

Referring now to FIGS. 5 and 6, a third embodiment of the invention is there shown generally at numeral 30 and includes a water insoluble inner pouch 36 preferably formed of Material D and filled with a hemostatic powder 40 formed of Material B and placed within a water soluble or water-swellable hemostatic outer pouch 32 formed of Material A. The inner pouch 36 is seamed and sealed closed at margins 38, while the outer pouch 32 is sealed around its margins 34. Again, this embodiment 30 may be placed with either side of the outer pouch 32 against the wound in the skin S.

Referring now to FIGS. 7 and 8, a fourth embodiment of the invention is there shown generally at numeral 50 and includes a panel of gauze material 54 formed of Material A backed by a moisture absorbing backing 52 formed of Material E in the form of a hydrophilic polyurethane foam. In use, this embodiment 50 would be applied against the skin S with the panel of gauze 54 thereagainst covering the wound.

In FIGS. 9 and 10, a fifth embodiment of the invention is shown generally at 60 and includes the same foam backing material 62 adhered against one surface of a pouch 64 formed of Material A and containing a quantity of particles 68 formed of Material B.

Referring now to FIGS. 11 and 12, a sixth embodiment of the invention is there shown generally at numeral 70 and includes a bottom layer of the water soluble, water-swellable material 74 formed of Material A, an intermediate layer 76 of hemostatic powder formed of Material B or a water absorbing material formed of Material C, and a top layer of moisture absorbing material 72 formed of Material E.

Referring now to FIGS. 13 and 13A, a seventh embodiment of the invention is there shown generally at numeral 80 including a two-stage approach to blood absorption and blood flow arrest from a wound. The first stage of blood absorption is performed by a quantity of a super-absorbing polymer formed of Material C (SAP) within an outer pouch 82 formed of Material D having a porous wicking bottom surface 84 which is positioned against the skin surrounding the wound. This SAP absorbs a large amount of blood which has exited the wound site and is laying atop the skin surface therearound.

A second stage of this device 80 includes a quantity of hemostatic powder 92 formed of Material B and separated from the SAP by an inner pouch 90 formed of Material D. The bottom surface 94 beneath the hemostatic agent particles 92 is formed of a water-soluble material made of Material A. This bottom panel 94 is seamed at 98 to the outer bottom panel 84, the water-soluble layer 94 being protectively covered with a removable aluminum foil panel 96 which remains in place until the device 80 is ready for use.

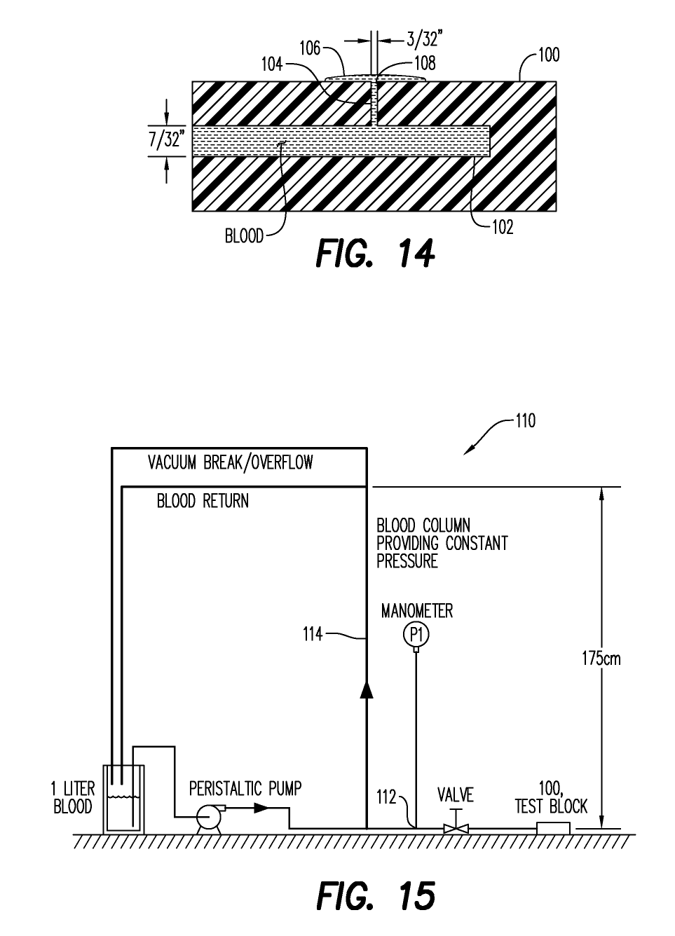

Referring additionally to FIG. 14, the test system includes an analytical balance (not shown), a timer, a test block 100, and a hemostasis apparatus shown generally at numeral 110. The hemostasis apparatus 110 includes a peristaltic pump which forces blood upwardly into a column 114 to provide a constant head pressure at the test block 100. The height of the column of blood 114 in relation to the test block 100 is proportional to the pressure at the outlet 108 of the test block 100 in passageway 104. The apparatus 110 must flow blood by the peristaltic pump at a sufficiently high flow rate of approximately 50-100 ml/min to demonstrate failure, but the velocity of the blood must be kept low to avoid lysing cells. A manometer P1 is used to maintain that consistent level of blood pressure.

The test block 100 is preferably formed of vinyl or PVC having an entry passageway 102 with a diameter of 7/16″. That passageway 102 is interconnected with a smaller passageway 104 having a diameter of 3/32″ leading to the open upper end 108. The sample 116 to be tested is applied over the open end 108 of this smaller passageway 104 atop the blood pool 106.

The manometer P1 is manufactured by Control Company under Model No. 06-664-19 having a pressure arrange capability of −15 to +15 psi. The peristaltic pump, made by Cole Parmer Easy Load II Head K-77200-60 is a constant on type pump to maintain the desired pressure level in the system, returning additional or overflow blood back into the one liter container of blood supply. Return and vacuum break tubing used in this apparatus 110 is ½″ diameter while the remainder of the tubing is ¼″, ⅛″ tubing being used between the valve and the test block 100 to minimize excess blood flow when test sample failure occurs.

The material used for the blood sample was Na EDTA treated blood or another form of stabilized whole bovine blood, 1.5 g Na EDTA/liter whole blood.

### Test Procedure

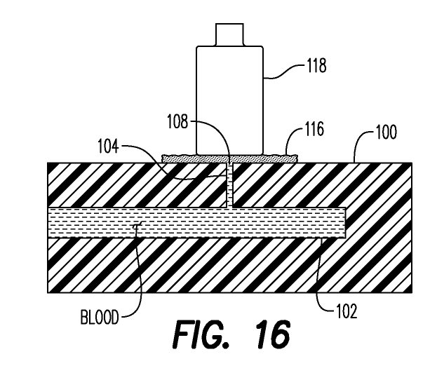

The preferred procedure for testing each test sample 106 for blood pressure to failure, i.e., when the test sample 116 fails to maintain the blood under pressure within the test block 100, includes the following steps:
1. With the valve closed and the pump running, record the blood pressure at the manometer P1 and record the height of the blood column.
2. With the valve closed, position a test block 100 at a height of the valve which is the same height 112 as the T connecting the manometer P1 to the blood flow line. Connect the opening of conduit 102 to the downstream side of the valve.
3. Open the valve slightly to allow a small quantity of blood 106 to flow to the surface of the valve 100 through the open end 108 of passageway 104, then close the valve.
4. Spread the blood 106 around the open end 108 of the passageway 104 to cover the area of at least ¼″ radius around the passageway 104 opening.
5. Cover this area around the opening of passageway 104 with approximately one gram of the hemostasis agent 116 to be tested, being sure to cover the outlet hole 108 and the immediate area therearound with the hemostasis agent 116 to a depth of at least 2 mm. A 1″×1″ dressing made with Material A may be used by itself or with one gram of hemostatic agent or absorbing agent on top of the dressing for testing.
6. Place a vial or bottle 128 over the test sample 126 as seen in FIG. 16, the vial or bottle 128 weighing 42 g and having a base diameter of approximately 1.25″.
7. With the valve remaining closed, keep the bottle or vial 128 in place for approximately 30 seconds, 60 seconds or 180 seconds, based upon the test being performed.
8. Open the valve to allow blood pressure into the test block 100, but do not remove the bottle or vial 118.
9. After 30 seconds, remove the bottle or vial 118 and wait for an additional 30 seconds.
10. If after 30 seconds, there is no sign of bleeding through the test sample 116, the test sample 116 has passed this test.
11. If the test fails, record the time to failure in seconds from the opening of the valve.

### Test Examples

An In Vitro Hemostasis Test Apparatus shown schematically in FIG. 15 was used to evaluate the efficiency to control bleeding of various dressings. The test provides a measure of seal strength as defined by the blood pressure at which a dressing fails.

### Example 1

A 1″×1″ dressing of a regenerated etherized and oxidized natural fiber cellulose (cellulose gauze) was evaluated and found to sustain 30 mm blood pressure for 30 seconds, but failed at higher pressure. While not constructed as a pouch, the cellulose gauze represents Material A described hereinabove.

### Example 2

PRO QR Powder, a mixture of potassium ferrate and hydrogen formed of sulfonated, 2% crosslinked polystyrene resin in accordance with U.S. Pat. No. 6,187,347 was evaluated, and found to sustain 100 mm blood pressure for 30 seconds but failed at higher pressure.

### Example 3

Cabloc 3050F powder was evaluated and found to sustain 30 mm blood pressure for 30 seconds, but failed at higher pressure. Cabloc 3050F is a super absorbing polymer (SAP) derived from partially neutralized crosslinked polyacrylic acid from Stewart Superabsorbents.

### Example 4

A reticulated (open-pore) polyurethane foam (urethane foam) from Crest Foam Industries with a porosity of 75 pores per inch and a thickness of ¼″ was tested in the test apparatus and found to not sustain any blood pressure. Blood was observed to leak thru the open cell foam.

### Example 5

The Hemostasis test was applied to a composite material consisting of cellulose gauze (same gauze used in Example 1) in direct contact with blood and urethane foam on top of the cellulose gauze. Blood pressure of 100 mm was sustained for 30 seconds compared of an expectation of 30 mm only from the sum of the pressures for each component.

### Example 6

The hemostasis test was applied to a composite material consisting of cellulose gauze (same gauze used in Example 1) in direct contact with blood and Cabloc 3050F SAP on top of the cellulose gauze. A pressure of 100 mm was sustained for 15 seconds compared to an expectation of 60 mm from the sum of pressures from the individual components.

### Example 7

The hemostasis test was applied to a composite material consisting of cellulose gauze (same gauze used in Example 1) in direct contact with blood and PRO QR Powder on top of the cellulose gauze. A pressure of 150 mm was sustained for 60 seconds compared to an expectation of 130 mm from the sum of pressures from the individual components.

Table 1 below summarizes the test results from Examples 1 to 7.

TABLE 1
Seal Strength of Dressings

| | | | Seal Strength |
|---|---|---|---|
| Ex- | | | (blood |
| ample | Dressing | Material | pressure mm) |
| 1 | Cellulose gauze | A | 30 |
| 2 | PRO QR Powder | B | 100 |
| 3 | Cabloc 3050F super absorbing | C | 30 |
| | polymer | | |
| 4 | Urethane Foam | E | 0 |
| 5 | Cellulose gauze + urethane foam | A&E | 100 |
| 6 | Cellulose gauze + Cabloc 3050F | A&C | 100 |
| 7 | Cellulose gauze + PRO QR Powder | A&B | 150 |

While a number of exemplary aspects and embodiments have been discussed above, those of skill in the art will recognize certain modifications, permeations and additions and subcombinations thereof. It is therefore intended that the following appended claims and claims hereinafter introduced are interpreted to include all such modifications, permeations, additions and subcombinations that are within their true spirit and scope.

1. A topically applied hemostatic wound dressing comprising:
a layer of water absorbing hydrophilic, anhydrous hemostatic material;
a quantity of loose hemostatic particles affixed to a non-skin-facing surface of said hemostatic material.

2. A topically applied hemostatic wound dressing as set forth in claim 1, wherein:
said hemostatic material is formed as a pouch of oxidized cellulose, polyvinylpyrrolidone, a copolymer of polyvinylpyrrolidone, alginate, cross-linked polyvinyl alcohol, or a copolymer of cross-linked polyvinyl alcohol;
said hemostatic particles being within said pouch.

3. A topically applied hemostatic wound dressing as set forth in claim 2, further comprising:
an outer pouch formed of water insoluble, water permeable material;
said powder being within said pouch, said pouch forming an inner pouch and being within said outer pouch.

4. A topically applied hemostatic wound dressing as set forth in claim 3, wherein:
said outer pouch is water impermeable except for having a water-wicking bottom skin-facing surface and substantially filled with a highly hydrophilic water absorbing material;
said inner pouch also being water impermeable except for having a central skin-facing surface formed of said hemostatic material affixed centrally of said outer skin-facing bottom surface;
wherein said water absorbing material absorbs a large quantity of blood surrounding a wound through said outer skin-facing surface when said central skin-facing surface of said inner pouch is placed against a heavily bleeding wound.

5. A topically applied hemostatic wound dressing as set forth in claim 2, further comprising:
a backing affixed to one side of said pouch which forms a non-skin-facing surface, said backing being formed of reinforcing moisture and blood absorbing foam or solid sheet material.

6. A topically applied hemostatic wound dressing as set forth in claim 2, wherein:
said hemostatic particles include powdered material that exerts a passive or an active mode of hemostatic action upon a bleeding wound.

7. A topically applied hemostatic wound dressing as set forth in claim 2, wherein:
said hemostatic particles are replaced with a highly hydrophilic water absorbing material.

8. A topically applied hemostatic wound dressing as set forth in claim 1, wherein:
said hemostatic material is formed as a pad;
said hemostatic material being affixed to one side of said pad.

9. A topically applied hemostatic wound dressing as set forth in claim 8, wherein:
said hemostatic material is formed of oxidized cellulose, polyvinylpyrrolidone, a copolymer of polyvinylpyrrolidone, alginate, cross-linked polyvinyl alcohol, or a cross-linked copolymer of polyvinyl alcohol.

10. A topically applied hemostatic wound dressing as set forth in claim 9, wherein:
said pad includes a skin adhesion-enhancing coating on a second side of said pad opposite to said hemostatic material.

11. A topically applied hemostatic wound dressing as set forth in claim 10, wherein:
said coating is a water sensitive coating taken from the group consisting of:
polyvinylpyrrolidone, a copolymer of polyvinylpyrrolidone, alginate, cross-linked polyvinyl alcohol and copolymers thereof, and polyvinyl alcohol.

12. A topically applied hemostatic wound dressing as set forth in claim 9, wherein:
said hemostatic particles are replaced with a highly hydrophilic water absorbing material.

13. A topically applied hemostatic wound dressing as set forth in claim 9, wherein:
said hemostatic particles include powdered material that exerts a passive or an active mode of hemostatic action upon a bleeding wound.

14. A topically applied hemostatic wound dressing as set forth in claim 9, further comprising:
a backing affixed to one side of said pouch which forms a non-skin-facing surface, said backing being formed of reinforcing moisture and blood absorbing foam or solid sheet material.

15. A topically applied hemostatic wound dressing comprising:
a backing formed of reinforced moisture and blood absorbing foam or solid sheet material;
a layer of water absorbing hydrophilic, anhydrous hemostatic material affixed to one side of said backing and forming a skin-facing surface.

16. A topically applied hemostatic wound dressing as set forth in claim 15, further comprising:
said hemostatic material is formed as a pouch of oxidized cellulose, polyvinylpyrrolidone, a copolymer of polyvinylpyrrolidone, alginate, cross-linked polyvinyl alcohol, or a copolymer of cross-linked polyvinyl alcohol;
said hemostatic particles being within said pouch.

17. A topically applied hemostatic wound dressing as set forth in claim 15, wherein:
said hemostatic particles are replaced with a highly hydrophilic water absorbing material.

18. A topically applied hemostatic wound dressing as set forth in claim 15, further comprising:
a quantity of loose hemostatic particles affixed between said backing and said hemostatic material and forming an intermediate layer therebetween.
